(19) United States
(12) Patent Application Publication
(10) Pub. No.: US 2025/0268264 A1
(43) Pub. Date: Aug. 28, 2025

(54) SYNERGISTIC FUNGAL COMBINATIONS AND METHODS OF USE

(71) Applicant: Thomas D. Johnson, Buffalo, SD (US)

(72) Inventor: Thomas D. Johnson, Buffalo, SD (US)

(21) Appl. No.: 18/588,232

(22) Filed: Feb. 27, 2024

(51) Int. Cl. A01N 63/38; A01N 63/30; A01P 21/00

(52) U.S. Cl. CPC A01N 63/38; A01N 63/30; A01P 21/00

(57) ABSTRACT

A synergistic fungal combinations for enhancement of one or more aspects of plant growth and decomposition includes a synergistic fungi composition comprising a Trichoderma virens strain, or a mutant thereof, and a Beauveria bassiana strain, or a mutant thereof. An amount of the composition that is applied to one or more of a seed, soil, an established plant, or crop residue from an untreated plant previously unexposed to the mixture of fungi that is sufficient to augment one or more of soil nutrient cycling including an increase in crop residue breakdown and release of nutrients from crop residue of the plant growing from the seed, the established plant, or an untreated plant previously unexposed to the mixture of fungi.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM.

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to fungal combinations and more particularly pertains to a new synergistic fungal combination that enhances one or more aspects of plant growth and decomposition. Soil carbon and organic matter are important components soil, allowing for the atmospheric deposition of carbon dioxide into the soil environment via photosynthesis and the decay of plant debris and waste. Carbon enters the soil by plant secretion of root exudates, decay of organic matter including plant and animal waste, and through the life cycle of soil microflora. Three fractions of soil organic matter are of particular importance and comprise up to 80% of total soil organic matter. These fractions are dissolved organic matter (5% with a turnover measured in days), particulate organic matter (2-25% with a turnover of 2-50 years) and humus (50% with a turnover of tens to hundreds of years). Soil organic matter also contains nitrogen, phosphorus, potassium, secondary and micronutrients, and it can retain water. Thus, soil organic matter is important for water and nutrient holding capacities within soil.

Decomposition of organic matter is determined by the organisms that are present in the soil, the physical environment, and the form(s) of the organic matter. In the breakdown of organic matter, carbon dioxide, water, nutrients, and organic compounds are released and humus is formed. Humus improves soil aggregation and soil stability, increases water holding capacity, improves the ability of the soil to attract nutrients, and reduces nutrient leaching.

In the breakdown of organic matter, root exudates comprised primarily of sugars, amino and organic acids, starches, and lipids are quickly consumed by bacteria in the rhizosphere. The dominant and insoluble fraction of soil organic matter consisting of cellulose, lignin and proteins degrades much more slowly. They are released over time to improve plant growth, and, once oxidized, the remaining residues are transformed into humus.

Trichoderma virens belongs to the Trichoderma genus classified as cellulolytic fungi from the phylum Ascomycota. Trichoderma spp. are widely used in enzyme production and in agriculture as either soil amendments or biopesticides. T. virens was originally placed within the Gliocladium genus due to morphological similarities but subsequently was reclassified to Trichoderma. Multiple T. virens species have been commercialized for agricultural use, including T. virens GL-21 (SoilGuard®, Certis Biologicals, Columbia, MD, USA), T. virens G41 (ATCC 20906), and T. virens GL-3 (ATCC 58678). Numerous other strains of T. virens have been isolated, primarily by USDA offices, and were found to have disease control properties or to elicit plant growth responses, including G-4 (ATCC MYA-297), G-6 (ATCC MYA-298), and G1-9 (ATCC 58677).

Beauveria bassiana is the most well-known entomopathogenic fungi. Due to its ability to infect a wide host of insects and low risk to humans, numerous B. bassiana strains have been developed as biopesticides. These biopesticides are registered for use as foliage sprays to control numerous insects on agronomically important crop species. B. bassiana controls insects by attachment to an insect cuticle, secretion of enzymes to penetrate the cuticle, and multiplication throughout the insect leading to insect death. B. bassiana are widely distributed in soils throughout the world. In addition to being an entomopathogenic fungi, B. bassiana have also been reported to be plant endophytes, although the degree of colonization and persistence of the organism within plant tissue varies greatly between individual trials, strains, and application methodologies.

The role that B. bassiana plays within the soil profile is poorly understood as research has been limited its interaction with plant pathogenic fungi, suppression of soil dwelling insects, and transfer of spores to the foliage of plants for colonization of leaf damaging insects. (Hatoon, H. et. al. “Role of microbes and soil organic carbon decomposition and maintenance of soil ecosystem” International Journal of Chemical Studies 2017; 5 (6): 1648-1656).

### (2) Description of Related Art Including Information Disclosed Under 37 CFR 1.97 and 1.98

The prior art relates to fungal combinations but which do not promote one or both of root colonization and endophytic colonization by B. bassiana.

### BRIEF SUMMARY OF THE INVENTION

An embodiment of the disclosure meets the needs presented above by generally comprising a synergistic fungi composition comprising a Trichoderma virens strain, or a mutant thereof, and a Beauveria bassiana strain, or a mutant thereof. An amount of the composition is applied to one or more of a seed, soil, an established plant, or crop residue from an untreated plant previously unexposed to the mixture of fungi that is sufficient to augment one or more of soil nutrient cycling including an increase in crop residue breakdown and release of nutrients from crop residue of the plant growing from the seed, the established plant, or an untreated plant previously unexposed to the mixture of fungi.

Another embodiment of the invention includes an amount of the composition being applied to one or more of a seed, soil, or an established plant that is sufficient to augment one or more of:
i. the growth of a plant growing from the seed or growth of the established plant,
ii. the health of the plant growing from the seed or health of the established plant,
iii. the quality of yield from the plant growing from the seed or quality of yield from the established plant,
iv. the quantity of yield from the plant growing from the seed or quantity of yield from the established plant, and
v. B. bassiana endophytic colonization of the plant growing from the seed or the established plant.

The composition can be applied to seeds, soil, or roots of an established plant to promote the growth of plants and/or to suppress plant diseases and pests. Methods of application include seed treatment, application to the soil at planting, application to the soil during the growing season, soil amendment, and application to crop residue from an untreated plant previously unexposed to the mixture of fungi. The composition can be applied alone, or for ease of application, it can be combined as a mixture with one or more of a fungicide, an insecticide, a plant growth promoting organism, a biopesticide a plant growth regulator, a fertilizer, or the like.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of the disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a microscope image of a Potato Dextrose Agar petri plate inoculated with T. virens GL-3 and B. bassiana GHA. The petri plate shows a novel response between a T. virens GL-3 colony and a B. bassiana GHA colony. There is an outgrowth of T. virens hyphae from the T. virens colony towards the B. bassiana colony. The margin (growth) of the T. virens colony is otherwise uninterrupted by hyphal extension suggesting that the T. virens growth is being elicited by the presence of the B. bassiana colony.

FIG. 2 is a photograph taken from a 2023 Nebraska corn field trial comparing a representative corn plant from the Untreated Control to a corn plant treated with T. virens GL-3 and B. bassiana GHA. Of note is the increase in root proliferation, stalk biomass, and leaf growth.

### DETAILED DESCRIPTION OF THE INVENTION

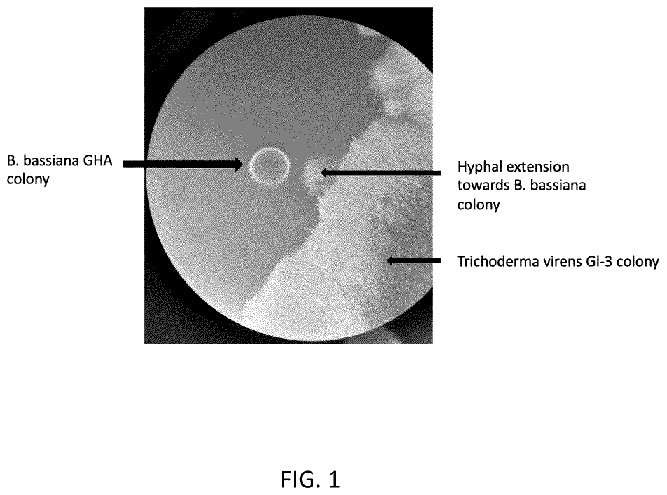

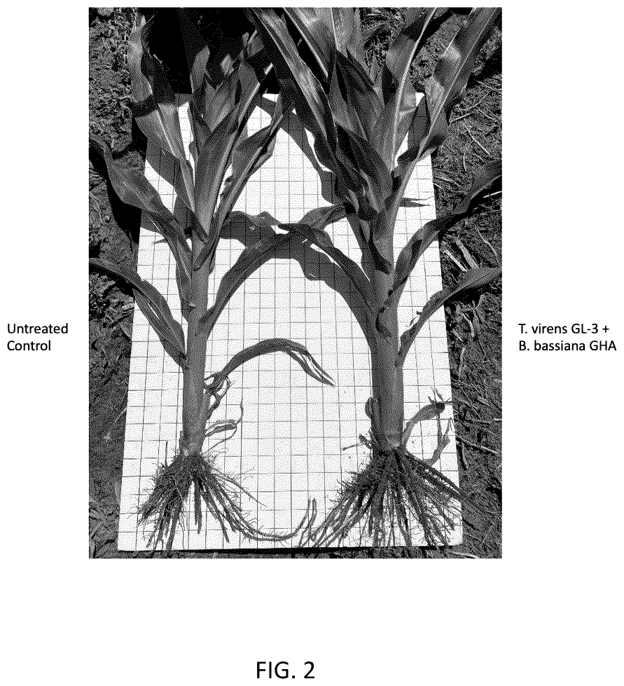

With reference now to the drawings, and in particular to FIGS. 1 through 2 thereof, a new fungal combination embodying the principles and concepts of an embodiment of the disclosure and generally designated by the reference numeral 10 will be described.

As best illustrated in FIGS. 1 through 2, the synergistic fungal combinations 10 generally comprises a synergistic fungi composition comprising a Trichoderma virens strain, or a mutant thereof, and a Beauveria bassiana strain, or a mutant thereof. Both organisms occur naturally in soils and plants throughout the world. New strains may be isolated in the future that fit within the scope of the disclosure.

The synergistic fungi composition comprises a mixture of T. virens and B. bassiana spores, though the organisms can also be applied sequentially to achieve comparable results. Depending on the specific formulation, the composition also may include sugars, such as maltodextrin, glucose, or the like, and seed flowability agents, such as talc, graphite, soy protein, synthetic and natural beeswax, or the like. The mixture of T. virens and B. bassiana can be applied as a dry powder directly to a seed, an established plant, or soil. The mixture of also can be suspended in water or an appropriate non-aqueous carrier and sprayed onto the seed, the established plant, or the soil.

The mixture of T. virens and B. bassiana spores can be applied to the soil via fertigation, applied into the soil at planting, or applied to seedling plants, and in particular to roots of the seedling plants, prior to their being transplanted into a field. The mixture of T. virens and B. bassiana also could comprise pesticides, such as fungicides, insecticides and biopesticides, other plant growth promoting organisms, fertilizers, plant growth regulators, and the like.

When using the mixture of T. virens and B. bassiana spores as a seed treatment, variations in seed shape, size and seeding rates for various crops need to be accounted for. Thus, different amounts of spores per seed are necessary to achieve appropriate coverage so as to assure seed viability and root colonization. Seed application rates most typically are T. virens ranging from 5E1 to 1E7 spores per seed and B. bassiana ranging from 1E2 and 1E8 colony forming units (cfu) per seed. Seed application rates may be T. virens ranging from 1E2 to 1E6 spores per seed and B. bassiana ranging from 5E2 to 1E7 spores per seed. In one example, seed application rates are T. virens ranging from 1.5E2 to 5E5 spores per seed and B. bassiana ranging from 1E3 to 5E6 spores per seed.

When using the mixture of T. virens and B. bassiana spores to treat seedling plants being grown in a greenhouse, shade house, outdoor environment, or the like, the seedling plants are treated with the mixture of T. virens and B. bassiana prior to being transplanted in a field. The T. virens and B. bassiana with T. virens are applied at a range of 5E1 to 1E8 spores per seedling plant and B. bassiana at a range of 1E2 to 5E8 spores per seedling plant. The T. virens and B. bassiana with T. virens may be applied at a range of 2E2 to 1E7 spores per seedling plant and B. bassiana at a range of 1E3 to 5E7 spores per seedling plant. A most preferred embodiment of the disclosure is a mixture comprising of T. virens and B. bassiana with T. virens applied at a range of 1E3 to 5E6 spores per plant and B. bassiana applied at a range of 5E3 to 2E7 spores per seedling plant.

The present invention also anticipates the mixture of T. virens and B. bassiana being added into hydroponic water, dipping the established plant in an aqueous solution containing the mixture of T. virens and B. bassiana, drenching the established plant with the mixture of T. virens and B. bassiana, and incorporating the mixture of T. virens and B. bassiana into the soil or a plant growing substrate.

Additionally anticipated by the present invention are treatment of the established plants by application of the mixture of T. virens and B. bassiana to a seed furrow, banding of the mixture of T. virens and B. bassiana to the side of the seed at planting, spraying the surface of the soil where an established plant is growing with of the mixture of T. virens and B. bassiana, and broadcast application the mixture of T. virens and B. bassiana onto the soil where established plants are growing or where plants will be grown. The mixture of T. virens and B. bassiana could also be incorporated onto dry granules or powders that are used to coat fertilizers including clay, sugars, compressed paper, humic acids, corn starch, and the like.

The present invention anticipates a per acre application rate of the mixture of T. virens and B. bassiana with T. virens ranging from 2E9 to 1E13 spores per acre and B. bassiana ranging from 1E10 to 5E13 spores per acre. The mixture of T. virens and B. bassiana may have a per acre application with T. virens ranging from 1E10 to 2E11 cfu per acre and B. bassiana ranging from 5E10 to 1E12 spores per acre. The mixture of T. virens and B. bassiana may have a per acre application with T. virens ranging from 2E10 to 1E11 spores per acre and B. bassiana ranging from 1E11 to 5E11 spores per acre.

We have determined that the synergistic fungi combination of B. bassiana and T. virens enables not only colonization of roots of plants by B. bassiana but also endophytic colonization by penetration of B. bassiana into the roots and translocation to the leaves of the plants.

Crop residue breakdown technologies in the market generally utilize bacteria and require the residues to have a low carbon to nitrogen ratio that typically is achieved through post-harvest application in the fall or early spring. We have determined that the synergistic fungi combination of B. bassiana and T. virens enables breakdown of crop residues having a low carbon to nitrogen ratio, a distinct advantage over the prior art. B. bassiana, through its endophytic colonization and selective cellulase, initiates breakdown of high carbon to nitrogen crop residues. T. virens produces additional cellulase and other enzymes to further degrade crop residues, thereby releasing valuable nutrients that would otherwise be tied up in the soil. Low molecular weight, relatively simple, carbohydrates are produced through the breakdown of cellulose in the crop residue allowing for the proliferation of bacteria that use that as a preferential food source. Because the crop residue breakdown process induced by the synergistic fungi combination happens over time, nutrients are released into the soil solution throughout a subsequent growing season.

It is anticipated that crop residue breakdown can be achieved using the application modalities discussed above and also through, and potentially enhanced by, application of the mixture of B. bassiana and T. virens to the crop residues post-harvest. For example, harvesting equipment could be fitted to apply the mixture of B. bassiana and T. virens to stover, straw, chafe, or the like, during harvesting or the mixture could be applied prior to the next planting.

Provided below are detailed examples of the synergistic fungi composition and methods for their use in soil nutrient cycling and in augmenting one or more of growth and health of the plant growing from the seed or of the established plant and quality or quantity of yield from the plant growing from the seed or of the established plant. These examples should not be viewed as limiting regarding compositions, methods of preparation and use, or plant species.

### I. Definitions

CHK stands for check and represents an untreated control in the trial. LSD stands for least significant difference and C.V. stands for Coefficient of Variance. In the footnotes, “a” denotes the corresponding value being significantly different from any other value that does not contain the letter “a” and “b” denotes the corresponding value being significantly different from any other value that does not contain the letter “b”.

For the strains utilized in the examples the following strains and sources were utilized: B. bassiana ANT-03 (BioCeres WP®, Anatis Bioprotection Inc., St-Jacques-le-Mineur, Quebec, Canada) standardized to 1E10 cfu/g; B. bassiana (BotaniGard 22WP®, Certis Biologicals, Columbia, MD) standardized to 2.2E10 cfu/g; T. virens GL-3 (ATCC 58678) grown on solid substrate and standardized to 1E10 cfu/g; T. virens G41 (ATCC 20906) grown on solid substrate and standardized to 1E10 cfu/g; Trichoderma harzianum T-22 (RootShield WP®, BioWorks, Inc., Victor, New York) standardized to 1E7 cfu/g; and Trichoderma asperellum T34 (Asperello®, BioBest USA Inc., Romulus, MI) standardized to 3E8 cfu/g.

### II. Plant Nutrient Cycling-Laboratory Substrate Decomposition Trials

### A. 2023 Corn Cob Substrate Decomposition

Table 1 presents the results of a laboratory trial testing decomposition of corn cob substrate. Corn cob has an exceptionally high carbon to nitrogen ration (approximately 100:1 and is an abundant agricultural crop residue. Interestingly, corn cob is used in cat/animal litter due to its high C: N ratio because the lack of nitrogen is a detriment to microbiological growth. 2.0 g of finely ground corn cob substrate was placed into 50 mL of RO Water in 50 mL autoclaved flasks. Flasks were inoculated with B. bassiana GHA (BotaniGard 22WP, Certis Biologicals, Columbia, MD), T. virens GL-3 (ATCC 58678), and a combination of both organisms. Flasks were maintained on an orbital shaker for 1 month before 1 mL samples were taken for plating, after which the remaining material was rinsed into weigh boats. Weigh boats were placed into a dehydrator for 10 hours before obtaining a final dry mass of the material. Each treatment was replicated four times.

Trial conclusions: The combination of T. virens and B. bassiana increased substrate breakdown by 0.04 g and 0.07 g over B. bassiana and T. virens individually, respectively. The combination of both organisms increased their respective growth rates, suggesting a synergistic and complimentary growth response when using complex carbohydrate residues as the sole source of growth.

TABLE 1

| | Substrate | Trichoderma | Beauvaria |
|---|---|---|---|
| Treatment | Mass (g) | (CFU/mL) | (CFU/mL) |
| B. bassiana GHA | 1.96 a | | 1.7E7 a |
| T. virens | 1.99 a | 1.2E6 b | |
| T. virens GL-3 + | 1.92 a | 2.0E6 a | 3.7E7 a |
| B. bassiana GHA | | | |
| LSD P = 0.05 | 0.102 | 5.47E5 | |
| C.V. | 3.28 | 23.05 | |

### B. 2023 Wheat Straw Substrate Decomposition

Table 2 presents the results of a laboratory trial testing decomposition wheat straw substrate. Wheat straw has a relatively high carbon to nitrogen ratio (approximately 80:1) and is a common crop residue in agricultural production. 1.6 grams of finely ground wheat straw substrate was placed into 20 mL of RO Water in 50 mL autoclaved flasks. Flasks were either uninoculated, inoculated with T. virens, or inoculated with a combination of the T. virens and B. bassiana. Flasks were maintained on an orbital shaker for 1 month before 1 mL samples were taken for plating, after which the remaining material was rinsed into weigh boats. Weigh boats were placed into a dehydrator for 10 hours before obtaining a final dry mass of the material. Each treatment was replicated four times.

Trial conclusions: The combination of T. virens and B. bassiana increased substrate breakdown by an additional 0.08 g (28.9%) over T. virens alone. The addition of B. bassiana to T. virens increased T. virens growth by 88% over T. virens alone.

TABLE 2

| | Substrate | Trichoderma |
|---|---|---|
| Treatment | Mass (g) | (CFU/mL) |
| Check | 1.57 a | |
| T. virens GL-3 | 1.37 ab | 2.15E7 |
| T. virens GL-3 + | 1.29 b | 4.05E7 |
| B. bassiana GHA | | |
| LSD P = 0.10 | 0.207 | |
| C.V. | 10.73 | |

### III. Replicated Field Trials

For the field trials, the mixtures of T. virens and B. bassiana were prepared from commercially available formulations of B. bassiana strain GHA and B. bassiana strain ANT03. T. virens strains GL-3 and G41 were obtained in pure spore, small batch quantities from a commercial producer of the strains. B. amyloliquefaciens was obtained as a pure, lyophilized spore from a commercial manufacturer.

### A. 2022 Nebraska Field Corn Seed Trial

Table 3 below presents the results of this field corn seed trial. Corn seed was treated with a standard fungicide/insecticide. For the treatments, the following were utilized as targeted cfu per seed: B. bassiana GHA: 2.25E6, B. bassiana ANT03: 4.5E5, and T. virens GL-3:1.12E5. The spores were applied to the seed as wettable powders, meaning they were first suspended in water and applied to the seed as a liquid slurry. Corn was seeded at a rate of 32,000 seeds per acre. Grower standard maintenance including fertilizer, irrigation, and weed and pest control were maintained throughout the trial. This trial was designed as a randomized complete block with each plot being 10 feet wide by 40 feet long and each treatment being replicated four times.

Trial conclusions: In this trial, the strains individually actually decreased yield relative to the untreated check. The combination of T. virens GL-3 and B. bassiana GHA was significantly different when compared to T. virens GL-3 alone. T. virens GL-3 and B. bassiana ANT-03 significantly increased yield when compared to the untreated control and when compared to the strains individually.

TABLE 3

| Treatment # | Treatment Name | Yield (Bushels/Acre) |
|---|---|---|
| 1 | CHK | 240.4 b, c |
| 2 | B. bassiana GHA | 239.0 b, c (−0.58%) |
| 3 | B. bassiana ANT03 | 233.0 c (−3.08%) |
| 4 | T. virens GL-3 | 232.3 c (−3.37%) |
| 5 | T. virens GL-3 + | 246.4 b (+2.50%) |
| | B. bassiana GHA | |
| 6 | T. virens GL-3 + | 262.8 a (+9.32%) |
| | B. bassiana ANT03 | |
| LSD P = 0.05 | | 9.4 |
| C.V. | | 2.57 |

### B. 2022 Nebraska Field Corn Seed Trial

Table 4 below presents the results of this field corn seed trial. Corn seed was treated with a standard fungicide/insecticide. For the treatments, the following were utilized as targeted cfu per seed: B. bassiana GHA: 2.25E6 and T. virens G41: 1.13E5. The spores were applied to the seed as wettable powders, meaning they were first suspended in water and applied to the seed as a liquid slurry. Corn was seeded at a rate of 34,000 seeds per acre. Grower standard maintenance including fertilizer, irrigation, and weed and pest control were maintained throughout the trial. This trial was designed as a randomized complete block with each plot being 10 feet wide by 40 feet long and each treatment was replicated four times.

Trial Conclusions: In this trial, the combination of T. virens strain G41 and B. bassiana strain GHA was significantly different from the untreated control and the strains individually.

TABLE 4

| Treatment # | Treatment Name | Yield (Bushels/Acre) |
|---|---|---|
| 1 | CHK | 205.9 c |
| 2 | T. virens G41 | 210.3 b (+2.14%) |
| 3 | B. bassiana GHA | 212.7 b (+3.30%) |
| 4 | T. virens G41 + | 217.9 a (+5.82%) |
| | B. bassiana GHA | |
| LSD P = 0.05 | | 4.42 |
| C.V. | | 1.31 |

### C. 2022 Illinois Soybean Seed Field Trial

Table 5 below presents the results of this soybean seed field trial. Soybean seed was treated with a standard fungicide/insecticide. For the treatments, the following were utilized as targeted cfu per seed: B. bassiana strain ANT03: 2.57E5 and T. virens G41: 6.43E4. The spores were applied to the seed as wettable powders, meaning they were first suspended in water and applied to the seed as a liquid slurry. Soybean was seeded at a rate of 140,000 seeds per acre. Grower standard maintenance including fertilizer, weed and pest control were maintained throughout the trial. This trial was designed as a randomized complete block with each plot being 10 feet wide by 30 feet long and each treatment was replicated four times.

Trial conclusions: In this trial, the combination of T. virens G41 and B. bassiana ANT03 was significantly different than the Untreated Control and B. bassiana ANT03 by itself, and numerically different than T. virens G41.

TABLE 5

| Treatment # | Treatment Name | Yield (Bushels/Acre) |
|---|---|---|
| 1 | CHK | 79.8 b |
| 2 | T. virens G41 | 82.0 ab (+2.76%) |
| 3 | B. bassiana ANT03 | 81.0 b (+1.50%) |
| 4 | T. virens G41 + | 84.1 a (+5.39%) |
| | B. bassiana ANT03 | |
| LSD P = 0.05 | | 2.85 |
| C.V. | | 2.18 |

### D. 2021 Multilocation Replicated Field Corn Seed Trial

Table 6 below presents the results of field trials at four different field sites. Each trial was planted and maintained using grower standard practices including seeding rates, fertilizer, irrigation, and weed and pest control. The trials each were designed as randomized replicated block with each plot being 10 feet wide by 40 feet long and each treatment was replicated four times. For the treatments, the following were utilized as targeted cfu per seed: B. bassiana strain GHA: 3.38E6, B. amyloliquefaciens: 3.13E6, and T. virens G41: 6.00E5. The spores were applied to the seed as a dry planter box treatment meaning they were dry coated onto the exterior of the seed prior to planting.

Trial Conclusions: The summary of the multilocation corn field trial showed that the only combination significantly different than the untreated control was the combination of T. virens G41 and B. bassiana GHA. This combination was numerically superior to B. bassiana by itself.

TABLE 6

| Treatment # | Treatment Name | Yield (Bushels/Acre) |
|---|---|---|
| 1 | CHK | 219.2 b |
| 2 | B. bassiana GHA | 224.6 ab (+2.46%) |
| 3 | B. bassiana GHA + | 222.0 b (+1.28%) |
| | B. amyloliquefaciens | |
| 4 | B. bassiana GHA + | 231.0 a (+5.38%) |
| | T. virens G41 | |
| 5 | T. virens G41 + | 223.5 b (+1.96%) |
| | B. amyloliquefaciens | |
| LSD P = 0.05 | LSD P = 0.05 | 6.84 |
| C.V. | | 1.98 |

### E. 2021 Multilocation Replicated Soybean Seed Field Trial

Table 7 below presents the results of these field trials at four different field sites. Each trial was planted and maintained using grower standard practices including seeding rates, fertilizer, irrigation, weed and pest control. The trials each were designed as randomized replicated block with each plot being 10 feet wide by 30 feet long and each treatment was replicated four times. For the treatments, the following were utilized as targeted cfu per seed: B. bassiana strain GHA: 1.93E6, B. amyloliquefaciens: 1.79E6, and T. virens G41: 3.43E5. The spores were applied to the seed as a dry planter box treatment meaning they were dry coated onto the exterior of the seeds prior to planting.

Trial Conclusions: The summary of the multilocation soybean field trial showed that the combination of B. bassiana GHA and T. virens G41 was significantly different than the untreated check, B. bassiana GHA, the combination of B. bassiana GHA and B. amyloliquefaciens, and numerically superior to the combination of T. virens G41 and B. amyloliquefaciens.

TABLE 7

| Treatment # | Treatment Name | Yield (Bushels/Acre) |
|---|---|---|
| 1 | CHK | 63.3 b |
| 2 | B. bassiana GHA | 60.5 c (−4.42%) |
| 3 | B. bassiana GHA + | 63.1 b, c (−0.32%) |
| | B. amyloliquefaciens | |
| 4 | B. bassiana GHA + | 66.2 a (+4.58%) |
| | T. virens G41 | |
| 5 | T. virens G41 + | 64.4 a, b (+1.74%) |
| | B. amyloliquefaciens | |
| LSD P = 0.05 | LSD P = 0.05 | 2.71 |
| C.V. | | 1.76 |

### F. 2022 Corn Field Seed Trial

Table 8 below presents the results of this field corn trial. Corn seed was treated with a standard fungicide/insecticide. For the treatments, the following were utilized as targeted cfu per seed: B. bassiana GHA: 2.25E6, T. virens G41: 1.13E5, Bacillus licheniformis: 2.25E6, and B. amyloliquefaciens: 2.25E6. The spores were applied to the seed as wettable powders, meaning they were first suspended in water and applied to the seed as a liquid slurry. Corn was seeded at a rate of 35,000 seeds per acre. Grower standard maintenance including fertilizer, weed and pest control were maintained throughout the trial. This trial was designed as a randomized complete block with each plot being 10 feet wide by 40 feet long and each treatment was replicated four times.

Trial Conclusions: In this trial, individually the bacterial species increased yield above the untreated control. However, when the organisms were combined together, a significant yield decrease occurred to yields similar to the Untreated Control. The addition of T. virens G41 and B. bassiana GHA to the Bacillus spp. seed treatment was significantly different to the Bacillus spp. combination and the Untreated Control. This synergistic response from the combinations of Bacillus spp. and the combination of Trichoderma and Beauveria is interesting as Bacillus spp. are the dominant Plant Growth Promoting Rhizobacteria and biopesticides in agriculture.

TABLE 8

| Treatment # | Treatment Name | Yield (Bushels/Acre) |
|---|---|---|
| 1 | CHK | 229.25 c |
| 2 | B. licheniformis | 234.25 b, c (+2.18%) |
| 3 | B. amyloliquefaciens | 245.75 a (+7.20%) |
| 4 | B. amyloliquefaciens + | 229.75 c (+0.22%) |
| | B. licheniformis | |
| 5 | B. amyloliquefaciens + | 236.25 b (+3.05%) |
| | B. licheniformis + | |
| | T. virens G41 + | |
| | B. bassiana GHA | |
| LSD P = 0.1 | | 5.431 |
| C.V. | | 4.19 |

### G. 2023 Soybean Field Seed Trial Summary

Table 9 below presents the results of this multi-location field soybean trial. Soybean seed was treated with a standard fungicide/insecticide. For the treatments, the following were utilized as target cfu/seed: B. bassiana ANT-03:1.30E5, B. bassiana GHA: 1.30E5 cfu/seed, T. virens GL-3 6.30E4 cfu/seed, and T. virens G41 6.30E4 cfu/seed. The spores were applied as a dry planter box treatment to the seed. Soybean was seeded at grower standard seeding rates. Grower standard maintenance including fertilizer, weed and pest control were maintained throughout the trial. Trials were designed as a randomized complete block with each plot being 10 feet wide by 40 feet long and each treatment was replicated four times. Each trial was replicated at 4 locations

Trial Conclusions: In this trial summary, all combinations of B. bassiana strains and T. virens strains statistically increased yield (LSD P=0.05) above the untreated control.

TABLE 9

| Treatment # | Treatment Name | Yield (Bushels/Acre) |
|---|---|---|
| 1 | CHK | 60.6 b |
| 2 | T. virens GL-3 + | 63.68 a (+4.6%) |
| | B. bassiana GHA | |
| 3 | T. virens G41 + | 63.85 a (+4.8%) |
| | B. bassiana GHA | |
| 4 | T. virens GL-3 + | 63.13 a (+3.7%) |
| | B. bassiana ANT-03 | |
| 5 | T. virens G41 + | 62.83 a (+3.2%) |
| | B. bassiana ANT-03 | |
| LSD P = 0.05 | | 1.910 |
| C.V. | | 1.97 |

In use, the synergistic fungi composition is applied to one or more of the seed, the soil, the roots of the established plant, or the crop residue from the untreated plant that was previously unexposed to the mixture of fungi. The mixture of B. bassiana and T. virens augments soil nutrient cycling from crop residue.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

1. A synergistic fungi system comprising:
a seed;
a mixture of fungi comprising:
a Trichoderma virens (T. virens) strain;
a Beauveria bassiana (B. bassiana) strain; and
the mixture being adjacent to the seed to augment soil nutrient cycling including an increase in crop residue breakdown and release of nutrients resulting in an improved crop yield.

2. (canceled)

3. (canceled)

4. (canceled)

5. The synergistic fungi system of claim 1, wherein the mixture of fungi comprises T. virens G41 and B. bassiana ANT-03.

6. The synergistic fungi system of claim 1, the mixture of fungi comprises T. virens G41 and B. bassiana GHA.

7. The synergistic fungi system of claim 1, wherein the mixture of fungi comprises T. virens GL-3 and B. bassiana ANT-03.

8. The synergistic fungi system of claim 1, wherein the mixture of fungi comprises T. virens GL-3 and B. bassiana GHA.

9. The synergistic fungi system of claim 1, wherein the mixture is generated by sequential application of the T. virens strain, and the B. bassiana strain.

10. The synergistic fungi system of claim 1, wherein the mixture having been applied adjacent to the seed at a targeted rate of 5E1 to 1E7 cfu per seed for the T. virens, and 1E2 and 1E8 colony forming units (cfu) per seed for the B. bassiana

11. The synergistic fungi system of claim 10, wherein the mixture having been applied adjacent to the seed at a targeted rate of 1E2 to 1E6 cfu per seed for the T. virens, and 5E2 to 1E7 cfu per seed for B. bassiana.

12. (canceled)

13. (canceled)

14. (canceled)

15. (canceled)

16. The synergistic fungi system of claim 1, wherein the seed is selected from one of a corn seed, a soybean seed, or a wheat seed.

17. (canceled)

18. (canceled)

19. (canceled)
